(19) United States
(12) Patent Application Publication
(10) Pub. No.: US 2025/0049947 A1
(43) Pub. Date: Feb. 13, 2025

(54) PAPN MUTANT, METHOD FOR SITE-DIRECTED MODIFICATION OF PAPN GENE AND USE THEREOF

(71) Applicant: AGSINO GENSOURCES CO., LTD., Shenzhen (CN)

(72) Inventors: Kui LI, Shenzhen (CN); Yulian MU, Shenzhen (CN); Lei HUANG, Shenzhen (CN)

(21) Appl. No.: 18/409,684

(22) Filed: Jan. 10, 2024

(30) Foreign Application Priority Data
Aug. 11, 2023 (CN) 202311010685.3

(51) Int. Cl. A61K 48/00; A61K 38/48; C12N 9/22; C12N 9/48; C12N 15/11; C12N 15/90

(52) U.S. Cl. CPC A61K 48/0033; A61K 38/48; C12N 9/22; C12N 9/485; C12N 15/11; C12N 15/907; C12Y 304/11002; C12N 2310/20; C12N 2800/80

(57) ABSTRACT

This description relates to a substance causing a mutation at position 727 in an amino acid residue of the pAPN protein in any of the following: 1) constructing a cell line with site-directed modification of the pAPN gene for non-disease diagnostic and therapeutic purposes; 2) preparing a cell with resistance to the porcine transmissible gastroenteritis virus; and so on. A system for site-directed modification of the pAPN gene may effectively cleave two target sites of the pAPN gene, thereby achieving the precise mutation at position 727 in an amino acid of pAPN. Based on the precise modification of the pAPN gene while capable of avoiding disruption or alteration of the normal expression of other amino acids in the pAPN, the physiological activity function of pAPN protein on the basis of resisting TGEV infection is retained.

### TECHNICAL FIELD

The present invention relates to the field of gene editing technology, in particular to a pAPN mutant and a system for site-directed modification of a pAPN gene and use thereof.

### BACKGROUND

Transmissible gastroenteritis (TGE) is a highly contagious intestinal disease mainly clinically characterized by death from severe diarrhea and rapid dehydration in infected piglets, which is a transmissible disease in pigs that must be strictly quarantined as required by the World Organization for Animal Health. The pathogen of this disease is the transmissible gastroenteritis virus (TGEV) in pigs, which infects piglets less than 2 weeks old with a very high mortality, especially piglets less than 10 days old with a fatality rate up to 100%. Therefore, TGE is considered as one of the important transmissible diseases that harm the pig feeding industry.

The invasion of TGEV into host cells is first achieved by binding the S protein of the virus to specific receptor protein molecules on the host cell membrane. Porcine aminopeptidase N (pAPN) is widely present on the surface of small intestinal epithelial cells and has a wide range of biological functions. pAPN is an important receptor for TGEV infection in the host cell. Meanwhile, sialic acid as a cofactor plays an important role in the adhesion of TGEV, because sialic acid helps to adhere more virions and promote the virus to cross the mucous layer of intestinal epithelium, so as to protect the virus from emulsification. pAPN as a proteolytic enzyme may hydrolyze peptides (such as neuropeptides and vasoactive peptides), as well as a signaling molecule involves in cell signaling and inflammatory chemotaxis, therefore, pAPN is associated with various physiological functions such as vascular growth and tumor invasion, and direct knockout of pAPN may affect other physiological functions of the body.

CRISPR/Cas9 gene editing technology, as a new generation of gene editing technology, can achieve precise gene editing, which provides a favorable tool for the construction of a genetically edited pig with TGEV resistance.

Therefore, it is particularly important to develop a precisely genetically edited pig with the precise mutation at key sites in pAPN gene, which can maintain normal expression of a pAPN protein and resist TGEV infection. This has important scientific and practical significance in pig disease resistance breeding.

### SUMMARY OF THE INVENTION

The objective of the present invention is to provide a pAPN mutant and a system for site-directed modification of a pAPN gene and use thereof.

In a first aspect, the present invention provides use of a substance causing a mutation at position 727 in an amino acid residue of a pAPN protein in any of the following:
1) constructing a cell line with site-directed modification of the pAPN gene for non-disease diagnostic and therapeutic purposes;
2) preparing a cell with resistance to the porcine transmissible gastroenteritis virus;
3) preparing a pig with resistance to the porcine transmissible gastroenteritis virus;
4) breeding the pig with resistance to the porcine transmissible gastroenteritis virus;
5) preparing a product for prevention or treatment of transmissible gastroenteritis in pigs, or preventing or treating transmissible gastroenteritis in pigs;
6) preparing a product for improvement of the resistance of a body or a body cell to the porcine transmissible gastroenteritis virus, or improving the resistance of a body or a body cell to the porcine transmissible gastroenteritis virus
7) constructing a cell model with resistance to the porcine transmissible gastroenteritis virus; and
8) constructing a pig model with resistance to the porcine transmissible gastroenteritis virus.

In the above, the substance causing a mutation at position 727 in an amino acid residue of the pAPN protein causes the mutation at position 727 in the amino acid residue of the pAPN protein, while not affecting the normal expression of the pAPN itself.

In the use described above, the mutation at position 727 in an amino acid residue of the pAPN protein is to mutate phenylalanine at position 727 in wild-type pAPN protein to alanine.

In the use described above, the body is a mammal, further a livestock, and still further a pig.

In the use described above, the substance causing a mutation at position 727 in an amino acid residue of the pAPN protein is a site-directed mutation system;
the site-directed mutation system is further a CRISPR system.

In the present invention, the CRISPR system comprises sgRNA1 or an expression cassette or vector expressing it, sgRNA2 or an expression cassette or vector expressing it, and a donor DNA for homologous recombination or an expression cassette or vector expressing it; and the sgRNA1 and sgRNA2 each target two respective target sites in pAPN gene, which can allow the genetically edited protein to target the sequence near the amino acid at position 727 in pAPN, but its specific sequence is not defined, as long as allowing for precise targeting function. The donor DNA contains a site-directed modified fragment of an amino acid at position 727 in pAPN that is used to replace the amino acid at position 727 in pAPN gene in order to mutate F727 in pAPN to A727, thereby achieving sequence recombination. The specific sequence of the donor DNA is not defined, as long as allowing for an amino acid mutation at position 727.

In an embodiment of the present invention, the target of sgRNA1 has a nucleotide sequence of SEQ ID NO: 1; and the target of sgRNA2 has a nucleotide sequence of SEQ ID NO: 2. The sgRNAs having sequences in SEQ ID NOs: 1 and 2 have more potent targeting and more precise modification.

In an embodiment of the present invention, the donor DNA for homologous recombination has a nucleotide sequence of SEQ ID NO: 3. The donor DNA can accurately replace the F727 (phenylalanine) encoded by pAPN gene with A727 (alanine).

In the CRISPR system for site-directed modification of the pAPN gene provided by the present invention, both sgRNA and sgRNA2 can target a fragment of interest that is enzymatically cleaved by the genetically edited protein. Sequence recombination is realized by using donor DNA again. The donor DNA is a replacement template for the modified target sequence of interest, which can specifically recognize the sequence near the amino acid site at position 727 encoded by pAPN gene under the guidance of sgRNA1 and sgRNA2. Based on the genetically edited protein, the target fragment is enzymatically cleaved and the donor DNA sequence is guided to replace an original homologous fragment in the cell, thereby achieving the purpose of precise modification of the amino acid at position 727 in pAPN.

Based on the precise modification of the amino acid at position 727 in pAPN on the basis of resisting TGEV infection while capable of avoiding disruption or alteration of the normal expression of other amino acids in pAPN, the CRISPR system provided by the present invention maximally retains the physiological activity function of the pAPN protein, and has advantages of wide applicability and high efficiency for gene editing and the like, which provides strong support for the preparation and breeding of new TGEV-resistant pig varieties with a single amino acid precise mutation in pAPN.

It should be noted that the CRISPR system for site-directed modification of the pAPN gene provided by the present invention can be used in any form acceptable in the art in combination with a genetically edited protein or a polynucleotides expressing the genetically edited protein. Based on the genetically edited protein, effectively enzymatic cleavage in various cells can be performed and the recombination of the cleaved sequence is guided, which has advantages of wide applicability and high efficiency for enzymatic cleavage. Types of genetically edited proteins are not defined by the present invention, as long as allowing for the genome editing function.

In an embodiment of the present invention, the CRISPR system comprises a first vector comprising an expression cassette for expressing the sgRNA1, a second vector comprising an expression cassette for expressing the sgRNA2, and a vector for expressing the donor DNA;
preferably, the first vector further comprises an expression cassette for a genetically edited protein;
preferably, the second vector further comprises an expression cassette for a genetically edited protein;
preferably, the genetically edited proteins expressed by the first and second vectors independently comprise, Cas9, Cas9n, Cpf1, or C2c2, respectively, but are not limited to this, and further independently preferably Cas9;
preferably, the backbones of the first and second vectors are independently derived from pX330, pX260, pX334, pX335, pX458, pX459, pX461, pX462, pX551, or pX552, respectively, but are not limited to this; and further independently preferably pX458.

Cas9 and pX458 have characteristics of wide universality, good versatility, and high product maturity, thus higher efficiency for enzymatic cleavage can be achieved by using pX458 as the backbone of the vector for gene editing.

In an optional embodiment, the single strands of oligonucleotides with sequences set forth in SEQ ID NOs: 4-5 and SEQ ID NOs: 6-7 is annealed, respectively, in order to form double strands, each of which is linked to the backbone of the enzymatically cleaved vector, and the first and second vectors are obtained by screening for positive clones.

In an embodiment of the present invention, the CRISPR system comprises pX458-pAPN-sgRNA-1 as a first vector, pX458-pAPN-sgRNA-2 as a second vector, and DNA Donor-727 as a vector expressing a donor.

In the above applications, the product is a kit or a drug.

In a second aspect, the present invention provides a construction of a cell model with resistance to the porcine transmissible gastroenteritis virus, comprising the steps of: expressing pAPN mutated protein in a starting cell to obtain a cell of interest, which is the cell model with resistance to the porcine transmissible gastroenteritis virus;

In an embodiment of the present invention, the starting cell is a porcine ileal epithelial cell.

The pAPN mutated protein is obtained by mutating phenylalanine at position 727 in wild-type pAPN protein to alanine, with no variation in other amino acid residues.

In the above, the wild-type pAPN protein has an amino acid sequence set forth in SEQ ID NO: 10 or corresponds to the pAPN protein set forth in SEQ ID NO: 10.

In a third aspect, the present invention provides a method for constructing a cell with pAPN protein mutation for non-diagnostic and therapeutic purposes, comprising the steps of: mutating phenylalanine at position 727 in wild-type pAPN protein to alanine in a cell of interest to obtain the cell with pAPN protein mutation.

In the above, phenylalanine at position 727 in wild-type pAPN protein is mutated to alanine in a cell of interest, specifically, the substance causing a mutation at position 727 in an amino acid residue of pAPN protein (i.e., a site-directed mutation system) is introduced into the cell of interest to obtain the cell with pAPN protein mutation;
preferably, the cell of interest comprises, but are not limited to, a porcine fibroblast, preferably a porcine ear fibroblast.
preferably, a method for introducing comprises electroporation or liposome transfection; further preferably electroporation.

In an optional embodiment, the preparation method further includes obtaining the cell with site-directed modification of a pAPN gene by screening and identification after the introduction operation. Preferably, the monoclonal cell is screened by flow cytometric sorting and identified whether it is the cell with site-directed modification at position 727 in pAPN, preferably by sequencing.

In an optional embodiment, DNA from the monoclonal cell can be extracted, followed by PCR amplification using primers set forth in SEQ ID NOs: 8-9 to obtain the amplified products, which can be sequenced to confirm whether the cell with precise modification.
preferably, the cell with precise modification of a pAPN gene is obtained by screening and identification after the introduction operation;
preferably, the screening comprises screening a monoclonal cell by flow cytometric sorting;
preferably, the identification comprises sequencing or PCR identification;
preferably, PCR identification is performed using primers set forth in SEQ ID NOs: 8-9.

In a fourth aspect, the present invention provides a method for constructing a genetically edited pig with pAPN protein mutation for non-diagnostic and therapeutic purposes, which is set forth in method 1 or method 2 as follows:
the method 1 comprises the steps of:
1) mutating phenylalanine at position 727 in pAPN protein to alanine in a porcine fibroblast ex vivo to obtain a cell of interest; and
2) transplanting the cell of interest as a donor cell for nuclear transplantation into a maternal pig by somatic cell nuclear transplantation to produce an offspring, which is the genetically edited pig with pAPN protein mutation;
the method 2 comprises the steps of:
microinjecting the substance causing a mutation at position 727 in an amino acid residue of a pAPN protein in the first aspect into a zygotic embryo in a pig to obtain a pAPN gene-modified embryo, followed by transplantation into the maternal body for pregnancy to obtain the genetically edited pig with pAPN protein mutation.

In the above, the genetically edited pig with pAPN protein mutation is a pig with a pAPN protein encoding gene on its genome that is the gene encoding the pAPN mutated protein;
the above pAPN mutated protein is obtained by mutating phenylalanine at position 727 in wild-type pAPN protein to alanine, with no variation in other amino acid residues.

The above porcine fibroblast is preferably a porcine ear fibroblast.

Preferably, a step of identification after birth is further comprised for the genetically edited pig;
preferably, the identification comprises sequencing or PCR identification;
preferably, PCR identification is performed using primers set forth in SEQ ID NOs: 8-9, and the amplified products are sequenced to confirm whether the pig has achieved precise modification.

In a fifth aspect, the present invention provides the substance causing a mutation at position 727 in an amino acid residue of a pAPN protein in the first aspect.

In a sixth aspect, the present invention provides a pAPN mutant that is obtained by mutating phenylalanine at position 727 in wild-type pAPN protein to alanine, with no variation in other amino acid residues.

In the above, the present invention does not limit whether the pAPN without mutation contains other mutation sites, so the precursor of the pAPN mutant provided by the present invention can be a wild-type pAPN or a pAPN mutant that has mutated at other sites based on the wild-type pAPN. The precursor of the pAPN mutant is considered a pAPN protein according to the general definition in the art.

In an optional embodiment, the amino acid sequence of wild-type pAPN is set forth in SEQ ID NO: 10 or corresponds to the pAPN protein set forth in SEQ ID NO: 10, specifically set forth in SEQ ID NO: 10, alternatively, comprises an amino acid sequence that is at least 80% identical with SEQ ID NO: 10, for example, but not limited to an amino acid sequence that is at least 80%, 85%, 90%, 95%, or 98% identical with SEQ ID NO: 10.

In a seventh aspect, the present invention provides a nucleic acid molecule encoding the pAPN mutant in the sixth aspect, or an expression cassette, a recombinant vector, or a recombinant cell containing the nucleic acid molecule.

“Nucleic acid molecule” herein refers to a polymeric form of a nucleotide including a ribonucleotide and/or a deoxyribonucleotide with any length. Examples of the nucleic acid molecule include, but are not limited to, single-stranded, double-stranded, or multi-stranded DNA or RNA, genomic DNA, cDNA, DNA-RNA heterozygote, or a polymer comprising purine and pyrimidine bases or other natural, chemical, or biochemical modified, unnatural, or derived nucleotide bases. The polynucleotide encodes the above pAPN mutant, which optionally encode a sense or antisense strand. The nucleic acid molecule can be naturally occurring, synthesized, recombinant, or any combination thereof. In an optional embodiment, the mutated base sequence at position 727 in the nucleic acid molecule encoding the mutation at position 727 in pAPN where an alanine is expressed has a sequence of GCC.

The site-directed modification of a pAPN gene can be achieved by the CRISPR system for precise modification of a pAPN gene provided by the present invention. This system can be used to construct a cell line with site-directed modification of a pAPN gene. Since F727 is an important amino acid site that affects the activity of a TGEV receptor, its point mutation can block the binding of pAPN and TGEV, so as to resist the infection of TGEV, thereby greatly enhancing the body's resistance to TGEV, and constructing a pig with transmissible gastroenteritis resistance. For ease of use, it is prepared into product forms such as a kit and the like.

According to another aspect of the present invention, it further provides a cell with pAPN mutation. The cell with pAPN mutation comprises a cell capable of expressing the pAPN mutant in the above embodiment; alternatively, the cell contains the polynucleotide encoding the pAPN mutant in the above embodiment, which can be expressed or not expressed in the cell with pAPN mutation, and it can only be replicated but not expressed in the cell with pAPN mutation. In an optional embodiment, the cell with pAPN mutation is prepared using the preparation method of the above cell with site-directed modification of a pAPN gene for non-disease diagnostic and therapeutic purposes.

In an eighth aspect, the present invention provides application of the pAPN mutant in the sixth aspect, or the nucleic acid molecule or the expression cassette, the recombinant vector, or the recombinant cell containing the nucleic acid molecule in the seventh aspect in any of the following:
1) constructing a cell line with site-directed modification of a pAPN gene for non-disease diagnostic and therapeutic purposes;
2) preparing a cell with resistance to the porcine transmissible gastroenteritis virus;
3) preparing a pig with resistance to the porcine transmissible gastroenteritis virus;
4) breeding the pig with resistance to the porcine transmissible gastroenteritis virus;
5) preparing a product for prevention or treatment of transmissible gastroenteritis in pigs, or preventing or treating transmissible gastroenteritis in pigs;
6) preparing a product for improvement of the resistance of a body or a body cell to the porcine transmissible gastroenteritis virus, or improving the resistance of a body or a body cell to the porcine transmissible gastroenteritis virus
7) constructing a cell model with resistance to the porcine transmissible gastroenteritis virus; and
8) constructing a pig model with resistance to the porcine transmissible gastroenteritis virus.

The present invention has the following beneficial effects compared with the prior art:
the pAPN mutant provided by the present invention with a mutation from phenylalanine to alanine at position 727 can maintain its normal expression, while reducing the specific binding between a host expressing the pAPN mutant and TGEV.

The CRISPR system for site-directed modification of a pAPN gene provided by the present invention comprises sgRNA1, gRNA2 and a donor DNA, which can effectively cleave two target sites in pAPN gene, thereby achieving precise mutation of the amino acid at position 727 in pAPN. Based on the precise modification of a pAPN gene while capable of avoiding disruption or alteration of the normal expression of other amino acids in pAPN, the present invention maximally retains the physiological activity function of a pAPN protein on the basis of resisting TGEV infection, and has advantages of wide applicability and high efficiency for gene editing and the like.

The preparation method of a cell with site-directed modification of a pAPN gene using the above CRISPR system has advantages of simple operation and low cost with accurate modification of the amino acid at position 727 in pAPN in the cell. The preparation method of a genetically edited pig obtained by using the cell with pAPN mutation has advantages of convenient operation and wide universality, and the prepared genetically edited pig with the amino acid mutation at position 727 have good TGEV resistance while retaining normal expression of a pAPN protein.

The technical solution of the present invention will be described clearly and completely by combining with examples below, and it is obvious that the described examples are part of the examples in the present invention, but not all of them. Based on the examples in the present invention, all other examples obtained by ordinary technicians in the art without creative efforts fall within the scope of protection of the present invention. Unless otherwise specified, the professional and scientific terms used herein have the same meanings as those familiar to skilled persons in the art. In addition, any method or material similar or equal to the recorded content can also be applied to the present invention.

### BRIEF DESCRIPTION OF DRAWINGS

FIG. 1 is an expression graph showing overexpressed pAPN proteins in porcine ileal epithelial cells with precise modification of amino acids at positions 726, 727 and 728 in pAPN proteins.

FIG. 2 is a graph of the results of fluorescence quantitative PCR (qPCR) for detection of the copy number of TGEV RNA in overexpressed porcine ileal epithelial cells infected with TGEV, in which amino acids at positions 726, 727 and 728 in pAPN proteins are precisely modified.

FIG. 3 is a graph of the results of Western Blot for detection of TGEV-N protein in overexpressed porcine ileal epithelial cells infected with TGEV, in which amino acids at positions 726, 727 and 728 in pAPN proteins are precisely modified.

FIG. 4 is a graph of the results of indirect immunofluorescence assay (IFA) for detection of pAPN and TGEV expressions in overexpressed porcine ileal epithelial cells infected with TGEV, in which amino acids at positions 726, 727 and 728 in pAPN proteins are precisely modified.

FIG. 5 is a pattern diagram of the precise mutation of the amino acid at position 727 in pig pAPN protein. The nucleotide sequence of E16 is shown in SEQ ID NO: 14, and the nucleotide sequence of HDR template is shown in SEQ ID NO: 15.

FIG. 6 is a graph of the results of sequencing for porcine ileal epithelial cell with precise modification of the amino acid at position 727 in pAPN protein. The nucleotide sequence of IPI-2I-WT is shown in SEQ ID NO: 16, and the nucleotide sequence of IPI-2I-727PE is shown in SEQ ID NO: 17.

FIG. 7 is an expression graph showing pAPN proteins in porcine ileal epithelial cells with precise modification of the amino acid at position 727 in pAPN protein.

FIG. 8 is a graph of the results of fluorescence quantitative PCR (qPCR) for detection of the copy number of TGEV RNA in porcine ileal epithelial cells infected with TGEV, in which the amino acid at position 727 in pAPN protein is precisely modified.

FIG. 9 is a graph of the results of Western Blot for detection of TGEV-N protein in porcine ileal epithelial cells infected with TGEV, in which the amino acid at position 727 in pAPN protein is precisely modified.

FIG. 10 is a graph of the results of indirect immunofluorescence assay (IFA) for detection of pAPN and TGEV expressions in porcine ileal epithelial cells infected with TGEV, in which the amino acid at position 727 in pAPN protein is precisely modified.

FIG. 11 is a graph of the results of pAPN enzyme activity for a porcine ileal epithelial cell with precise modification of the amino acid at position 727 in pAPN protein.

FIG. 12 is a graph of the results of sequencing for porcine fibroblasts with precise modification of the amino acid at position 727 in pAPN protein. The nucleotide sequence of PEF-WT is shown in SEQ ID NO: 18, and the nucleotide sequence of PEF-727PE is shown in SEQ ID NO: 19.

FIG. 13 is a graph of the results of sequencing for a genetically edited pig with precise modification of the amino acid at position 727 in pAPN protein. The nucleotide sequence of WT is shown in SEQ ID NO: 20, and the nucleotide sequence of PIG-727PE is shown in SEQ ID NO: 21.

### DETAILED DESCRIPTION

The experimental methods used in Examples below are conventional methods unless otherwise specified.

The materials, reagents and the like used in Examples below can be obtained commercially unless otherwise specified.

The present invention is further illustrated by specific examples below, but it should be understood that these examples are intended only for more detailed illustration purposes and should not be understood to limit the present invention in any form.

### Main Reagents:

Collagenase type IV for isolation of porcine ear fibroblasts were purchased from sigma; DMEM, FBS, PS, NEAA, and Glutamine for cell culture were all purchased from Gibco; the DNA kit for extracting cells and ear tissues was purchased from Tiangen Biotech Co., Ltd.; primers were synthesized by Beijing Tsingke Biotech Co., Ltd.; and the KOD FX PCR enzyme for PCR was purchased from TOYOBO.

### Main Instruments:

CO2 incubator (Thermo Scientific, 3111); clean bench (AIRTECH, SW-CJ-1FD); inverted fluorescence microscope (ZEISS, observerA1); PCR instrument (BIO-RID, C1000 Touch); gel imaging system (BIO-RID, Universal Hood II); micromanipulation system (Eppendorf, Celltramvario); flow cytometric sorter (BD, Aria III).

Wild-type porcine ileal epithelial cells (IPI-2I-WT) and porcine ileal epithelial cells with pAPN gene knockout (Immortal Pig Intestinal-21 Knock Out, IPI-2I-KO) in Examples below were both documented in the reference of “Xu Changjiang, Wang Xiaopeng, Xu Kui et al. Establishment of pAPN gene knockout IPI-21 cell lines Mediated by CRISPR/Cas9 System. China Animal Husbandry and Veterinary Medicine, 2021, 48 (7): 2282-2290.”.

The precise modification in Examples below referred to a site-directed mutation, specifically an amino acid site-directed mutation.

### Example 1. Obtaining Overexpressed Porcine Ileal Epithelial Cells with Precise Modification of Amino Acids at Positions 726, 727 and 728 in pAPN and Verifying their Anti-TGEV Function

I. Establishment of Overexpressed Porcine Ileal Epithelial Cells with Precise Modification of Amino Acids at Positions 726, 727 and 728 in pAPN and Detection of pAPN Expressions in them

The CDS sequence of wild-type pAPN gene had a nucleotide sequence of SEQ ID NO: 12;

The CDS sequence of pAPN gene with precise modification of the amino acid at position 726 was obtained by mutating CTC at positions 2176-2178 in the gene encoding wild-type pAPN protein (SEQ ID NO: 12) to GCC (i.e., the leucine codon CTC at position 726 in wild-type pAPN protein was mutated to the alanine codon GCC), with no variation in other nucleotides.

The CDS sequence of pAPN gene with precise modification of the amino acid at position 727 was obtained by mutating TTC at positions 2179-2181 in the gene encoding wild-type pAPN protein (SEQ ID NO: 12) to GCC (i.e., the phenylalanine codon TTC at position 727 in wild-type pAPN protein was mutated to the alanine codon GCC), with no variation in other nucleotides, specifically, the CDS sequence of pAPN gene with precise modification of the amino acid at position 727 had a nucleotide sequence of SEQ ID NO: 13.

The CDS sequence of pAPN gene with precise modification of the amino acid at position 728 was obtained by mutating CAA at positions 2182-2184 in the gene encoding wild-type pAPN protein (SEQ ID NO: 12) to GCC (i.e., the glutamine codon CAA at position 728 in wild-type pAPN protein was mutated to the alanine codon GCC), with no variation in other nucleotides.
1. CDS sequences of wild-type pAPN gene, as well as pAPN gene with precise modification of amino acids at positions 726, 727 (having the nucleotide sequence of SEQ ID NO: 13) and 728 were separately linked to the polyclonal sites (BamHI and XbaI enzyme cleavable sites) of PLVX backbone vectors (Y0025620-1, Beijing Tsingke Biotech Co., Ltd.), named PLVX-WT, PLVX-726, PLVX-727 and PLVX-728, respectively, followed by plasmid sequencing for future use after expansion.
2. On the day before electrotransfection, porcine ileal epithelial cells with pAPN gene knockout (Immortal Pig Intestinal-2I Knock Out, IPI-2I-KO; with no expression of pAPN gene) were recovered into 10 cm dishes, and the cell transfection could be performed until to about 80% confluence of cells.
3. PLVX-WT, PLVX-726, PLVX-727, PLVX-728 and PLVX empty vector were electrotransfected into IPI-2I-KO cells, named IPI-2I-WTOE, IPI-2I-726OE, IPI-2I-727OE, IPI-2I-728OE and IPI-2I-Vector, respectively. Meanwhile, the untransfected IPI-2I-KO cells were used as the Mock group.

Cells were collected at 12 h after transfection, followed by extraction of cell proteins for detection of the expression of pAPN by Western blot. The antibody was an APN polyclonal antibody (ABclonal, A5662).

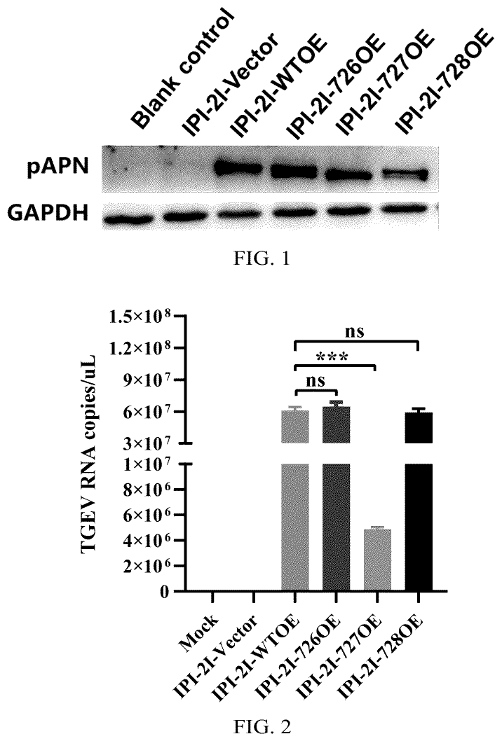

The results of pAPN protein detection were shown in FIG. 1, indicating that the overexpressed vectors were successfully transfected into IPI-2I-KO cells in the IPI-2I-WTOE, IPI-2I-726OE, IPI-2I-727OE and IPI-2I-728OE groups, and pAPN was expressed normally.

This result indicated that porcine ileal epithelial cells with precise modification and overexpression of pAPN had been successfully obtained, which might be used as donor cells for subsequent TGEV infection experiments.

II. Anti-TGEV Function Verification of Overexpressed Porcine Ileal Epithelial Cells with Precise Modification of Amino Acids at Positions 726, 727 and 728 in pAPN

IPI-2I-WTOE, IPI-2I-726OE, IPI-2I-727OE, IPI-2I-728OE and IPI-2I-Vector cells obtained by transfection in step I above were tested for TGEV infection with the specific steps as follows:
1. IPI-2I-WTOE, IPI-2I-726OE, IPI-2I-727OE, IPI-2I-728OE and IPI-2I-Vector cells were inoculated with TGEV virus strains (MOI=1), respectively. Meanwhile, IPI-2I-KO cells without virus inoculation were used as the Mock group.
2. Cells were collected at 12 h after infection, and washed 4-5 times with PBS, followed by extraction of RNA for detection of the copy number of TGEV virus in cells by qPCR. Meanwhile, IPI-2I-KO cells without virus inoculation were used as the Mock group.

The qRT-PCR results were shown in FIG. 2, indicating that there were no significant changes in the copy number of TGEV genomic RNA in IPI-2I-726OE and IPI-2I-728OE cells (P>0.05), while the copy number of TGEV genomic RNA in IPI-2I-727OE cells was significantly reduced (*** P<0.001), compared with IPI-2I-WTOE cells.
3. Cells were collected at 12 h after infection, followed by extraction of cell proteins for detection of the expression of TGEV-N protein by Western blot, meanwhile, IPI-2I-KO cells without virus inoculation were used as the Mock group.

The expression of TGEV-N protein was shown in FIG. 3, indicating that there was no significant change in the expression of TGEV-N protein in IPI-2I-726OE and IPI-2I-728OE cells, while the expression of TGEV-N protein in IPI-2I-727OE cells was significantly reduced, compared with IPI-2I-WTOE cells.
4. Cells were collected at 12 h after infection, followed by detection of TGEV infection in cells by indirect immunofluorescence assay (IFA).

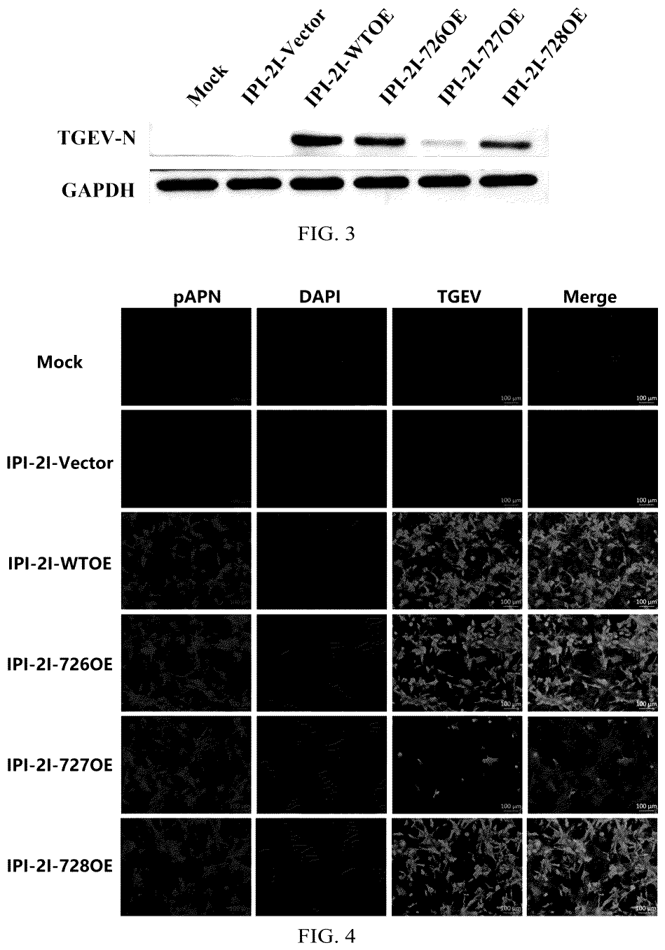

The IFA detection results were shown in FIG. 4, indicating that IPI-2I-WTOE cells were infected with a large number of TGEVs after virus inoculation; and there was no significant change in the amount of TGEV infection in the IPI-2I-726OE and IPI-2I-728OE groups, while the amount of TGEV infection in IPI-2I-727OE cells was significantly reduced compared with IPI-2I-WTOE cells.

In summary, the results showed that the overexpressed porcine ileal epithelial cells with precise modification of amino acids at positions 726 and 728 in pAPN could not effectively resist TGEV infection, indicating that the amino acids at positions 726 and 728 in pAPN were not the key sites for TGEV infection; but overexpressed porcine ileal epithelial cells with precise modification of amino acids at position 727 in pAPN could effectively resist TGEV infection, indicating that the amino acid at positions 727 in pAPN was the key site for TGEV infection.

### Example 2. Construction of an Expression Vector for a System of Precise Site-Directed Modification at Position 727 in pAPN

The wild-type pAPN protein had an amino acid sequence of SEQ ID NO: 10.

The wild-type pAPN protein was modified by site-directed mutation to obtain mutants as follows:
pAPN protein mutant 727, which was obtained by mutating phenylalanine at position 727 in the amino acid sequence of wild-type pAPN protein to alanine, with no variation in other amino acid residues;
the gene encoding pAPN protein mutant 727, which was obtained by mutating TTC at positions 2179-2181 in the gene encoding wild-type pAPN protein (SEQ ID NO: 12) to GCC (i.e., the phenylalanine codon TTC at position 727 in wild-type pAPN protein was mutated to the alanine codon GCC), with no variation in other nucleotides.

The specific preparation method was as follows:

I. Design of sgRNA Sequence and Construction of a Vector
1. sgRNA sequence synthesis

The targeting site that was close to the amino acid site encoding 727 and had a higher score was selected with porcine pAPN gene as the sequence of interest using the sgRNA analysis tool CRISPOR (crispor.tefor.net), as shown below:

Sequence encoding pAPN-sgRNA-1 was:

(SEQ ID NO: 1)
CTAGAAATACCTCAGGAAGC

Sequence encoding pAPN-sgRNA-2 was:

(SEQ ID NO: 2)
GTTTCGAAATGTTGGAAGAG.

Synthesis of complementary paired oligonucleotide sequences for pAPN-sgRNA-1 and pAPN-sgRNA-2 sequences:

pAPN-sgRNA-1-F:
(SEQ ID NO: 4)
caccgCTAGAAATACCTCAGGAAGC;
pAPN-sgRNA-1-R:
(SEQ ID NO: 5)
aaacGCTTCCTGAGGTATTTCTAGc;
pAPN-sgRNA-2-F:
(SEQ ID NO: 6)
caccGTTTCGAAATGTTGGAAGAG;
pAPN-sgRNA-2-R:
(SEQ ID NO: 7)
aaacCTCTTCCAACATTTCGAAAC.

2. Construction of the first and second vectors
(1) The pAPN-sgRNA-1-F/pAPN-sgRNA-1-R and pAPN-sgRNA-2-F/pAPN-sgRNA-2-R obtained in step I were treated at 98° C. for 10 min, respectively, and then cooled to room temperature in natural environment for annealing to obtain pAPN-sgRNA-1 and pAPN-sgRNA-2 annealed products.
(2) The pX458 backbone vector containing Cas9 sequence (addgene, 48138) was cleaved with the restrictive endonuclease BbsI at 37° C. for 2 h, followed by recovery of the linearized fragments that was the backbone of the linearized vector by gel cutting.
(3) The above annealed double-stranded fragments were separately linked with the backbone of the linearized vector at 16° C. for 1 h, cooled in ice bath for 30 min, followed by heat shock for 45 s, and then transformed into Top10 or DH5a competent cells, which were coated and grown on LB plates containing ampicillin, and single colonies were picked on the next day for culturing and sequencing. Sequencing primers were as follows:

U6-FWD:
(SEQ ID NO: 11)
GAGGGCCTATTTCCCATGATT.

3. The first (pX458-pAPN-sgRNA-1 plasmid) and second (pX458-pAPN-sgRNA-2 plasmid) vectors were extracted and cultured after sequences being correct, and frozen at −20° C. for subsequent cell transfection. The plasmid extraction was carried out using the EndoFree Plasmid Maxi Kit (CoWin Biotech, CW2104M).

The first and second vectors were named pX458-pAPN-sgRNA-1 and pX458-pAPN-sgRNA-2, respectively.

### II. Design of Donor DNA Sequence and Construction of Donor-727 Vector

1. Design of donor DNA sequence

Based on the sgRNA sequence, a donor DNA named pAPN-dsODN-727 with its specific sequence set forth in SEQ ID NO: 3 was designed for precise modification of the amino acid at position 727 in pAPN.

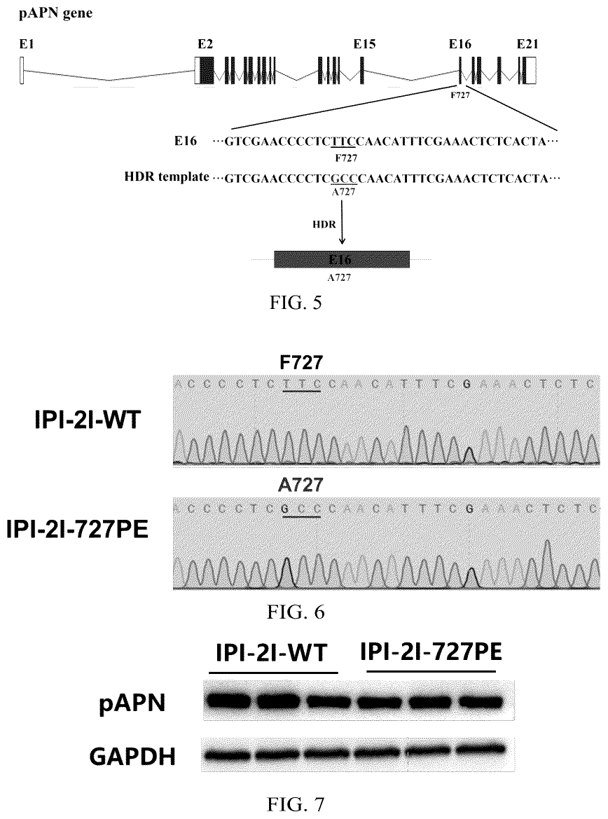

The pAPN-dsODN-727 as a double stranded Donor sequence replaced the wild-type pAPN gene sequence, so that F727 in wild-type pAPN protein was successfully replaced with A727. A pattern diagram of precise mutation of single amino acid at position 727 in pAPN protein was set forth in FIG. 5.
2. Construction of Donor-727 vector

The donor DNA pAPN-dsODN-727 obtained in step I was linked to the polyclonal site (EcoRV s enzyme cleavable site) of the PUC57 backbone vector (Genscript Biotech Corporation, SD1176) for sequencing validation, and the correctly sequenced recombinant plasmid was then amplified to obtain the vector Donor-727 for subsequent cell transfection. The plasmid extraction was carried out using the EndoFree Plasmid Maxi Kit (CoWin Biotech, CW2104M).

### Example 3. Obtaining Porcine Ileal Epithelial Positive Cells with Precise Modification of the Amino Acid at Position 727 in pAPN and Verifying their Anti-TGEV Function

I. Obtaining Porcine Ileal Epithelial Positive Cells with Precise Modification of the Amino Acid at Position 727 in pAPN
1. Wild-type porcine ileal epithelial cells (IPI-2I-WT) were recovered into a 10 cm dish, and the cell transfection could be performed until to about 80% confluence of cells. 5 μg of pX458-pAPN-sgRNA-1 plasmids prepared in Example 2, 5 μg of pX458-pAPN-sgRNA-2 plasmids prepared in Example 2 and 5 μg of Donor-727 plasmids prepared in Example 2 were co-transfected into IPI-2I-WT with steps following strictly to the instructions of the Basic Primary Nucleofector Kit (Lonza, VPI-1002).
2. Cells were digested and collected into a tube for flow cytometry at 48 h after electroporation. Individual GFP positive cells were sorted using a flow cytometric sorter and cultured in a 96 well plate with refreshment of the culture medium every 3 days. Cells were passaged to a 48 well plate for culture until filling the 96 well plate, and then a portion of cells were taken for genome extraction and genotype identification until filling the 48 well plate.
3. The picked monoclonal cells were identified with specific steps of: the extracted cell genomic DNA as a template, using pAPN-TY-F2 (SEQ ID No: 8) and pAPN-TY-R2 (SEQ ID No: 9) for PCR amplification to obtain PCR products with a size of 1443 bp.

The amplification condition for PCR was as follows: 94° C. for 5 min; 94° C. for 30 s, 62.6° C. for 30 s, 68° C. for Imin 40 s, 34 cycles; 72° C. for 5 min. PCR products were sequenced by Beijing TianyiHuiyuan Company. Based on the results of sequencing, IPI-21 (IPI-2I-727PE) cells with precise modification of the amino acid at position 727 in pAPN protein were screened for subsequent experiments.

The results of sequencing showed that multiple strains of IPI-2I-727PE cells with precise modification of the amino acid at position 727 in pAPN protein were successfully obtained in this example, and the results of sequencing for the positive cells were set forth in FIG. 6.

Compared with IPI-2I-WT cells, IPI-2I-727PE cells were obtained by replacement the phenylalanine codon TTC at the position 727 in pAPN protein with the alanine codon GCC on the genome of IPI-2I-WT cells.

II. Anti-TGEV Function Verification of Porcine Ileal Epithelial Positive Cells with Precise Modification of the Amino Acid at Position 727 in pAPN

IPI-2I-727PE cells obtained in above step I were tested for TGEV infection with specific steps of:
1. IPI-2I-727PE and IPI-2I-WT cells were inoculated with TGEV virus (MOI=1), respectively. Meanwhile, IPI-2I-WT cells without virus inoculation were used as the Mock group.
2. Cells were collected at 12 h after infection, followed by extraction of cell proteins for detection of the expression of pAPN by Western blot. The antibody was an APN polyclonal antibody (ABclonal, A5662).

The results showed that the expression level of pAPN protein in IPI-2I-727PE cells was comparable to that in IPI-2I-WT cells (FIG. 7). This indicated that the precise modification at position 727 of the amino acid in pAPN did not affect the normal expression of the protein.
3. Cells were collected at 12 h after infection, and washed 4-5 times with PBS, followed by extraction of RNA for detection of the copy number of TGEV virus in cells by qPCR. Meanwhile, IPI-2I-WT cells without virus inoculation were used as the Mock group.

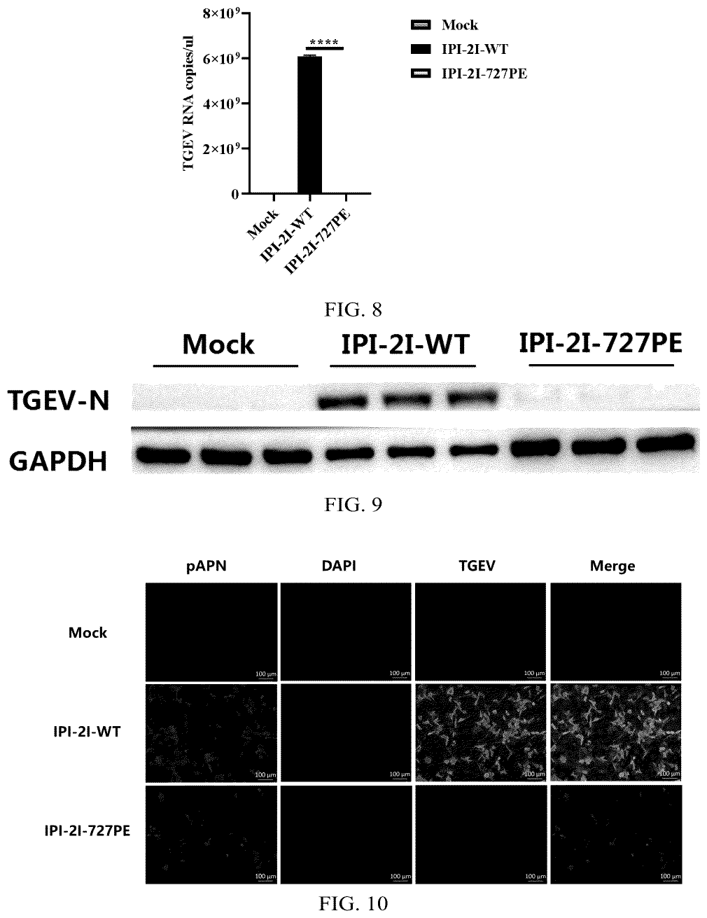

The qRT-PCR results were shown in FIG. 8, indicating that the copy number of TGEV genomic RNA in IPI-2I-727PE cells was significantly reduced (**** P<0.001), compared with IPI-2I-WT cells.
4. Cells were collected at 12 h after infection, followed by extraction of cell proteins for detection of the expression of TGEV-N protein by Western blot. Meanwhile, IPI-2I-WT cells without virus inoculation were used as the Mock group.

The expression of TGEV-N protein was shown in FIG. 9, indicating that the expression of TGEV-N protein in IPI-2I-727PE cells was significantly reduced, compared with IPI-2I-WT cells.
5. Cells were collected at 12 h after infection, followed by detection of TGEV infection in cells by indirect immunofluorescence assay (IFA). Meanwhile, IPI-2I-WT cells without virus inoculation were used as the Mock group.

The IFA detection results were set forth in FIG. 10, indicating that IPI-2I-WT cells were infected with a large number of TGEVs after virus inoculation; and the amount of TGEV infection in IPI-2I-727PE cells was significantly reduced compared with IPI-2I-WT cells.

In summary, the above results showed that the porcine ileal epithelial cells with a mutation from phenylalanine to alanine at position 727 in pAPN could effectively resist TGEV infection, indicating that the position 727 in pAPN was a key site for TGEV infection, and the mutation from phenylalanine to alanine at position 727 in pAPN could effectively resist TGEV infection.

### Example 4. Determination of Enzyme Activity in Porcine Ileal Epithelial Positive Cells with Precise Modification of the Amino Acid at Position 727 in pAPN

IPI-2I-WT, IPI-2I-727PE, and IPI-2I-KO cells were cultured for 24 h, followed by harvesting cells, and the cell suspension was washed with PBS. Then, 5×10⁵ cells in 200 μL of PBS per well were suspended in a 96 well plate, with addition of a substrate of l-leucine p-nitroaniline (Sigma Aldrich, L9125) to the final concentration of 1.6 mM, followed by incubation at 37° C. for 1 h, and the absorbance at 405 nm was measured every 15 min using a microplate reader to detect pAPN enzyme activity.

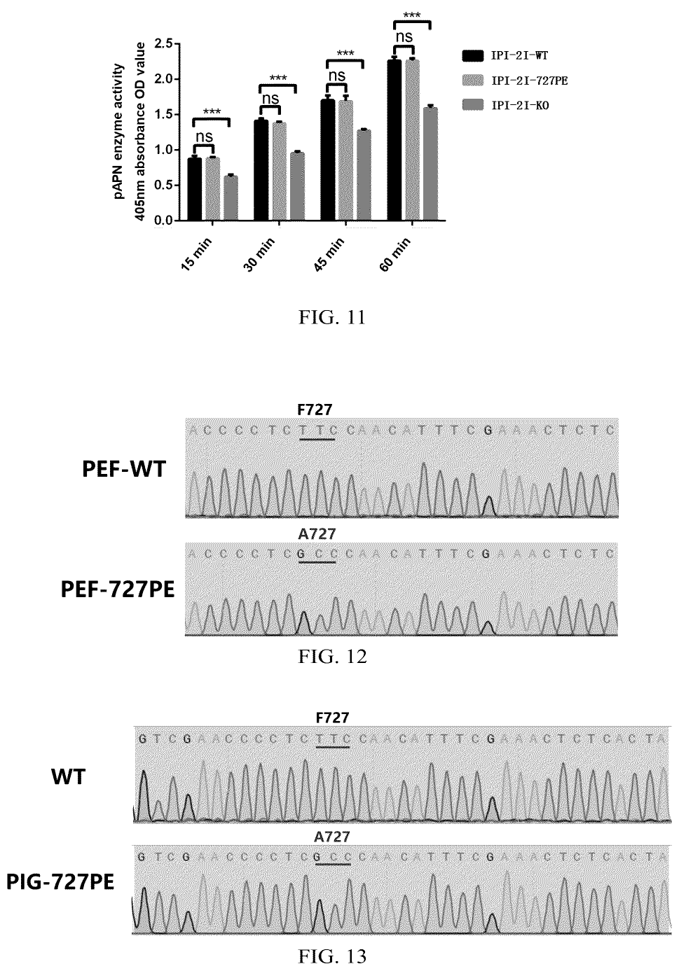

The results of pAPN enzyme activity detection were shown in FIG. 11, indicating that compared with IPI-2I-WT cells, there was no significant difference in pAPN enzyme activity of cells in the IPI-2I-727PE group at reaction times of 15 min, 30 min, 45 min, and 60 min, while the enzyme activity of IPI-2I-KO cells was significantly reduced (*** P<0.001).

Therefore, the precise modification of the amino acid at position 727 in pAPN (phenylalanine mutated to alanine) did not affect the normal enzyme activity of the protein.

### Example 5. Obtaining the Monoclone from Porcine Fibroblasts with Precise Modification of the Amino Acid at Position 727 in pAPN

### I. Preparation of Porcine Ear Fibroblasts

Ear derived tissues from healthy 1 month old Large White purebred pigs were collected. The ear tissue samples were firstly soaked in 75% alcohol and washed with PBS (Invitrogen, C10010500BT). Then, the epidermal tissues were scraped using a surgical blade, and the remaining parts were cut into pieces using ophthalmic scissors in a cell culture dish. Finally, the tissue blocks were transferred to a T-25 cell culture flask (Eppendorf, 0030710126) and placed in a CO2 incubator for cultivation, while observing the growth of cells around the tissue blocks. Medium was refreshed every 2 days. The cells were frozen for later use until growing to a density of about 80%-90% to obtain primary porcine ear fibroblasts.

### II. Cell Transfection

The primary porcine ear fibroblasts obtained above were recovered into a 10 cm dish at the day before transfection, and when the cells reached a confluence of about 80%, the porcine ear fibroblasts to be transfected could be obtained for cell transfection.
5 μg of pX458-pAPN-sgRNA-1 plasmids prepared in Example 2, 5 μg pX458-pAPN-sgRNA-2 plasmids and 5 μg Donor-727 plasmids were co-transfected into porcine ear fibroblasts with steps following strictly to the instructions of the Basic Primary Fibroblasts Nucleofector Kit (Lonza), and then the electrotransfected cells were transferred to a 6-well plate for culture.

### III. Flow Cytometric Sorting and Passage of Monoclonal Cells

Cells were digested and collected into a tube for flow cytometry at 48 h after electroporation. Individual GFP positive cells were sorted using a flow cytometric sorter and cultured in a 96 well plate with refreshment of the culture medium every 3 days. Cells were passaged to a 48 well plate for culture until filling the 96 well plate, and then a portion of cells were taken for genome extraction and genotype identification until filling the 48 well plate.

### IV. Obtaining Monoclonal Cells

Identification of picked monoclonal cells: the extracted DNA genome was amplified with the upstream and downstream primers set forth in SEQ ID NO: 8 and SEQ ID NO: 9 to obtain a 1443 bp fragment, using the extracted cell genome as a template. The amplification condition was as follows: 94° C. for 5 min; 94° C. for 30 s, 62.6° C. for 30 s, 68° C. for Imin 40 s, 34 cycles; 72° C. for 5 min. PCR products were sequenced by Beijing TianyiHuiyuan Company.

Based on the results of sequencing, porcine fibroblasts with a mutation from phenylalanine to alanine of the amino acid at position 727 in pAPN protein were selected as positive cells, which could serve as donor cells for nuclear transplantation.

The sequencing results from some positive cells were shown in FIG. 12, indicating that multiple strains of positive cells were successfully obtained in the present example, which were porcine fibroblasts with precise modification of the amino acid at position 727 in pAPN (phenylalanine codon TTC mutated to alanine codon GCC), named PEF-727PE.

### Example 6. Preparation of a Genetically Edited Pig with Precise Modification of the Amino Acid at Position 727 in pAPN by Somatic Cell Nuclear Transplantation Technology

The porcine fibroblast PEF-727PE with precise modification of the amino acid at position 727 in pAPN obtained in Example 5 were used as donor cells for nuclear transplantation, and the enucleated porcine oocytes matured in vitro for 40 h were used as recipient cells for nuclear transplantation. The donor cells for nuclear transplantation were transferred into the oocytes, which were electrically fused and activated to construct recombinant cloned embryos. The well-developed cloned recombinant embryos were selected and surgically transplanted into the uterus of naturally estrous multiparous white sows for pregnancy. In this process, steps of surgical embryo transplantation were as follows: the recipient sow was anesthetized by intravenous injection of Zoletil with a dosage of 5 mg/kg body weight. After anesthesia, the recipient sows were moved to an operating rack for supine fixation, followed by respiratory anesthesia (with a concentration of 3% to 4% isoflurane). An about 10 cm long of surgical incision was made at the midline of the abdomen of the recipient sow to expose ovaries, fallopian tubes, and uterus. An embryo transplantation glass tube was used to enter about 5 cm along the fimbria of the fallopian tubes, and the well-developed cloned recombinant embryos were transplanted to the junction between the ampulla and isthmus of the fallopian tubes. Embryos were regularly observed by the technicians after transplantation, and the pregnancy statuses of the recipient sows were examined by B-type ultrasound.

Ear tissues were cut from piglets after birth, followed by extraction of genomic DNA, which was amplified by PCR using the above nucleotide sequences of SEQ ID NO: 8 and SEQ ID NO: 9, and the products from PCR amplification were sequenced for genotype detection.

The sequencing results showed that the genetically edited pig (PIG-727PE) with a mutation from phenylalanine to alanine of the amino acid at position 727 in pAPN protein was successfully obtained, which was the genetically edited pig with precise modification of the amino acid at position 727 in pAPN. The sequencing results of some genetically edited pigs were shown in FIG. 13, indicating that compared with the wild-type pAPN protein, the genetically edited pig with precise modification of the amino acid at position 727 in pAPN protein that was a pig of interest with resistance to TGEV infection had a mutation from phenylalanine codon TTC to alanine codon GCC at position in pAPN protein, with no variation in other amino acid residues.

Finally, it should be noted that the above examples are only used to illustrate the technical solutions of the present invention and not to limit it. Although the present invention has been described in detail with reference to the above examples, it should be understood by persons of ordinary skill in the art that the technical solutions recorded in the above examples may be modified or equivalently replaced some or all of the technical features. However, these modifications or replacements should not cause the essence of the corresponding technical solutions to deviate from the scope of the technical solutions of various examples in the present invention.

1. The sequence encoding pAPN-sgRNA-1:
(SEQ ID NO: 1)
CTAGAAATACCTCAGGAAGC
2. The sequence encoding pAPN-sgRNA-2:
(SEQ ID NO: 2)
GTTTCGAAATGTTGGAAGAG.
3. Specific sequence of pAPN-dsODN-727:
(SEQ ID NO: 3)
CCTTTGAGCACAGTCTGGCCTTGTGCGAGGCCTTTAGCCTCTGGCCT
CTTGCTCCTGTAGCCATTAGCTCTTGCTACATCTGCCCACCCACATCAG
AGGCTCCATGGGTCTCCAGATGACTCAGGCATGAGTCTCTTCTTTGAAG
CTATTTTTAGGGCTGCATCCTCGGCATGTGGAGGTTCCCAAGCTAGGGG
TTGAATCGGAGCTGTAGCCGCCAGCCTACACCACAGCCACAGCAACACG
GGATCCGAGCCACATCTGCGACCTACACCACAGCTCACAGCAATGCCAG
ATCCTTAACCCACTGAGTGGGGCCAGGGTTGAACCCATGTCCTCATGTT
TCCCAGTCAGATTCGTTTCTGCTGTGCCATGACGGGAACTCTGGAACTT
CCTCTTTGAAGCTCTTTATGTTTTGTTCTTGTTTTTTGTTTTTGTTTTT
CTAGAAATACCTCAGGAAGCAAGTCGAACCCCTCGCCCAACATTTCGAA
ACTCTCACTAAAAACTGGACCGAGCGCCCAGAAAATCTGATGGACCAGT
GAGTATGAGCTCGCTTGGTCTGGAGATCATGGGTGGTGCAGGTAGCCTG
ACCTGGGGGCCCATAGCAAGTCCAGCAGCATCCTCTCTGGAGCTCCCAA
CTCCTGGCCGGACCAGGGCCACAGTCAGGGAGAGCGACCCCTCCCAACC
CCACTCCCGGCCCCAGGAGTAGGGACTCTGCTCTGAGGCTCTGTGTGGC
CTATGAACCATCTGGCCTCTTTGGGCAAAGGACCAAACTGAACCTCTGA
GGGTCCCTCACCCGCATGGTGAGGTTCTAGGTGTTAAAGCTGGGGCTGG
AGCCTGTGCCAGCCCTCCCCAGGCTGCCCAAGGGCAAGAAGCAAAGAAG
GGAACCCAAAGGTGGCTGGTGGGCTATACCTGCAGAGTGCGGGTCTGCC
TCCCTGTTGGGAGTTGTGTGTCAGCAGGGGAGTCTTGGTCAGCGTCAGG
TCCAGGCGTGCTGACAGAGTGT
4. pAPN-sgRNA-1-F:
(SEQ ID NO: 4)
caccgCTAGAAATACCTCAGGAAGC;
5. pAPN-sgRNA-1-R:
(SEQ ID NO: 5)
aaacGCTTCCTGAGGTATTTCTAGc;
6. pAPN-sgRNA-2-F:
(SEQ ID NO: 6)
caccGTTTCGAAATGTTGGAAGAG;
7. pAPN -sgRNA-2-R:
(SEQ ID NO: 7)
aaacCTCTTCCAACATTTCGAAAC;
8. pAPN-TY-F2 sequence:
(SEQ ID NO: 8)
CAAGGATTTGTGGAGGAGAA;
9. pAPN-TY-R2 sequence:
(SEQ ID NO: 9)
GCTGAGCGGAGTTTGTCG;
10. Wild-type amino acid sequence of pAPN:
(SEQ ID NO: 10)
MAKGFYISKALGILGILLGVAAVATIIALSVVYAQEKNKNAEHVPQAPT
SPTITTTAAITLDQSKPWNRYRLPTTLLPDSYNVTLRPYLTPNADGLYI
FKGKSIVRFICQEPTDVIIIHSKKLNYTTQGHMVVLRGVGDSQVPEIDR
TELVELTEYLVVHLKGSLQPGHMYEMESEFQGELADDLAGFYRSEYMEG
NVKKVLATTQMQSTDARKSFPCFDEPAMKATFNITLIHPNNLTALSNMP
PKGSSTPLAEDPNWSVTEFETTPVMSTYLLAYIVSEFQSVNETAQNGVL
IRIWARPNAIAEGHGMYALNVTGPILNFFANHYNTPYPLPKSDQIALPD
FNAGAMENWGLVTYRENALLFDPQSSSISNKERVVTVIAHELAHQWFGN
LVTLAWWNDLWLNEGFASYVEYLGADHAEPTWNLKDLIVPGDVYRVMAV
DALASSHPLTTPAEEVNTPAQISEMFDSISYSKGASVIRMLSNFLTEDL
FKEGLASYLHAFAYQNTTYLDLWEHLQKAVDAQTSIRLPDTVRAIMDRW
TLQMGFPVITVDTKTGNISQKHFLLDSESNVTRSSAFDYLWIVPISSIK
NGVMQDHYWLRDVSQAQNDLFKTASDDWVLLNINVTGYFQVNYDEDNWR
MIQHQLQTNLSVIPVINRAQVIYDSENLATAHMVPVTLALDNTLFLNGE
KEYMPWQAALSSLSYFSLMFDRSEVYGPMKKYLRKQVEPLFQHFETLTK
NWTERPENLMDQYSEINAISTACSNGLPQCENLAKTLFDQWMSDPENNP
IHPNLRSTIYCNAIAQGGQDQWDFAWGQLQQAQLVNEADKLRSALACSN
EVWLLNRYLGYTLNPDLIRKQDATSTINSIASNVIGQPLAWDFVQSNWK
KLFQDYGGGSFSFSNLIQGVTRRFSSEFELQQLEQFKKNNMDVGFGSGT
RALEQALEKTKANIKWVKENKEVVLNWFIEHS;
11. U6-FWD sequence of the sequencing primer:
(SEQ ID NO: 11)
GAGGGCCTATTTCCCATGATT;
12. CDS sequence of wild-type pAPN gene:
(SEQ ID NO: 12)
ATGGCCAAGGGATTCTACATTTCCAAGGCCCTGGGCATCCTGGGCAT
CCTCCTCGGCGTGGCGGCCGTGGCCACCATCATCGCTCTGTCTGTGGTG
TACGCCCAGGAGAAGAACAAGAATGCCGAGCATGTCCCCCAGGCCCCCA
CGTCGCCCACCATCACCACCACAGCCGCCATCACCTTGGACCAGAGCAA
GCCGTGGAACCGGTACCGCCTACCCACAACGCTGTTGCCTGATTCCTAC
AACGTGACGCTGAGACCCTACCTCACTCCCAACGCGGATGGCCTGTACA
TCTTCAAGGGCAAAAGCATCGTCCGCTTCATCTGCCAGGAGCCCACCGA
TGTCATCATCATCCATAGCAAGAAGCTCAACTACACCACCCAGGGGCAC
ATGGTGGTCCTGCGGGGCGTGGGGGACTCCCAGGTCCCAGAGATCGACA
GGACTGAGCTGGTAGAGCTCACTGAGTACCTGGTGGTCCACCTCAAGGG
CTCGCTGCAGCCCGGCCACATGTACGAGATGGAGAGTGAATTCCAGGGG
GAACTTGCCGACGACCTGGCAGGCTTCTACCGCAGCGAGTACATGGAGG
GCAACGTCAAAAAGGTGCTGGCCACGACACAGATGCAGTCTACAGATGC
CCGGAAATCCTTCCCATGCTTTGACGAGCCAGCCATGAAGGCCACGTTC
AACATCACTCTCATCCACCCTAACAACCTCACGGCCCTGTCCAATATGC
CGCCCAAAGGTTCCAGCACCCCACTTGCAGAAGACCCCAACTGGTCTGT
CACTGAGTTCGAAACCACACCTGTGATGTCCACGTACCTTCTGGCCTAC
ATCGTGAGCGAGTTCCAGAGCGTGAATGAAACGGCCCAAAATGGCGTCC
TGATCCGGATCTGGGCTCGGCCTAATGCAATTGCAGAGGGCCATGGCAT
GTATGCCCTGAATGTGACAGGTCCCATCCTAAACTTCTTTGCCAATCAT
TATAATACACCCTACCCACTCCCCAAATCCGACCAGATTGCCTTGCCCG
ACTTCAATGCCGGTGCCATGGAGAACTGGGGGCTGGTGACCTACCGGGA
GAACGCGCTGCTGTTTGACCCACAGTCCTCCTCCATCAGCAACAAAGAG
CGAGTTGTCACTGTGATTGCTCACGAGCTGGCCCACCAGTGGTTTGGCA
ACCTGGTGACCCTGGCCTGGTGGAATGACCTGTGGCTGAATGAGGGCTT
TGCCTCCTATGTGGAGTACCTGGGTGCTGACCACGCAGAGCCCACCTGG
AATCTGAAAGACCTCATCGTGCCAGGCGACGTGTACCGAGTGATGGCTG
TGGATGCTCTGGCTTCCTCCCACCCGCTGACCACCCCTGCTGAGGAGGT
CAACACACCTGCCCAGATCAGCGAGATGTTTGACTCCATCTCCTACAGC
AAGGGAGCCTCGGTTATCAGGATGCTCTCCAACTTCCTGACTGAGGACC
TGTTCAAGGAGGGCCTGGCGTCCTACTTGCATGCCTTTGCCTATCAGAA
CACCACCTACCTGGACCTGTGGGAGCACCTGCAGAAGGCTGTGGATGCT
CAGACGTCCATCAGGCTGCCAGACACTGTGAGAGCCATCATGGATCGAT
GGACCCTGCAGATGGGCTTCCCCGTCATCACCGTGGACACCAAGACAGG
AAACATCTCACAGAAGCACTTCCTCCTCGACTCCGAATCCAACGTCACC
CGCTCCTCAGCGTTCGACTACCTCTGGATTGTTCCCATCTCATCTATTA
AAAATGGTGTGATGCAGGATCACTACTGGCTGCGGGATGTTTCCCAAGC
CCAGAATGATTTGTTCAAAACCGCATCGGACGATTGGGTCTTGCTGAAC
ATCAACGTGACAGGCTATTTCCAGGTGAACTACGACGAGGACAACTGGA
GGATGATTCAGCATCAGCTGCAGACAAACCTGTCGGTCATCCCTGTCAT
CAATCGGGCTCAGGTCATCTACGACAGCTTCAACCTGGCCACTGCCCAC
ATGGTCCCTGTCACCCTGGCTCTGGACAACACCCTCTTCCTGAACGGAG
AGAAAGAGTACATGCCCTGGCAGGCCGCCCTGAGCAGCCTGAGCTACTT
CAGCCTCATGTTCGACCGCTCCGAGGTCTATGGCCCCATGAAGAAATAC
CTCAGGAAGCAGGTCGAACCCCTCTTCCAACATTTCGAAACTCTCACTA
AAAACTGGACCGAGCGCCCAGAAAATCTGATGGACCAGTACAGTGAGAT
TAATGCCATCAGCACTGCCTGCTCCAATGGATTGCCTCAATGTGAGAAT
CTGGCCAAGACCCTTTTCGACCAGTGGATGAGCGACCCAGAAAATAACC
CGATCCACCCCAACCTGCGGTCCACCATCTACTGCAATGCCATAGCCCA
GGGCGGCCAGGACCAGTGGGACTTTGCCTGGGGGCAGTTACAACAAGCC
CAGCTGGTAAATGAGGCCGACAAACTCCGCTCAGCGCTGGCCTGCAGCA
ACGAGGTCTGGCTCCTGAACAGGTACCTGGGTTACACCCTGAACCCGGA
CCTCATTCGGAAGCAAGACGCCACCTCCACTATTAACAGCATTGCCAGC
AATGTCATCGGGCAGCCTCTGGCCTGGGATTTTGTCCAGAGCAACTGGA
AGAAGCTCTTTCAGGACTATGGCGGTGGTTCCTTCTCCTTCTCCAACCT
CATCCAGGGTGTGACCCGAAGATTCTCCTCTGAGTTTGAGCTGCAGCAG
CTGGAGCAGTTCAAGAAGAACAACATGGATGTGGGCTTCGGCTCCGGCA
CCCGGGCTCTGGAGCAAGCCCTGGAGAAGACCAAGGCCAACATCAAGTG
GGTGAAGGAGAACAAGGAGGTGGTGTTGAATTGGTTCATAGAGCACAGC
TAA;
13. CDS sequence of pAPN gene with precise
modification of the amino acid at position 727:
(SEQ ID NO: 13)
ATGGCCAAGGGATTCTACATTTCCAAGGCCCTGGGCATCCTGGGCAT
CCTCCTCGGCGTGGCGGCCGTGGCCACCATCATCGCTCTGTCTGTGGTG
TACGCCCAGGAGAAGAACAAGAATGCCGAGCATGTCCCCCAGGCCCCCA
CGTCGCCCACCATCACCACCACAGCCGCCATCACCTTGGACCAGAGCAA
GCCGTGGAACCGGTACCGCCTACCCACAACGCTGTTGCCTGATTCCTAC
AACGTGACGCTGAGACCCTACCTCACTCCCAACGCGGATGGCCTGTACA
TCTTCAAGGGCAAAAGCATCGTCCGCTTCATCTGCCAGGAGCCCACCGA
TGTCATCATCATCCATAGCAAGAAGCTCAACTACACCACCCAGGGGCAC
ATGGTGGTCCTGCGGGGCGTGGGGGACTCCCAGGTCCCAGAGATCGACA
GGACTGAGCTGGTAGAGCTCACTGAGTACCTGGTGGTCCACCTCAAGGG
CTCGCTGCAGCCCGGCCACATGTACGAGATGGAGAGTGAATTCCAGGGG
GAACTTGCCGACGACCTGGCAGGCTTCTACCGCAGCGAGTACATGGAGG
GCAACGTCAAAAAGGTGCTGGCCACGACACAGATGCAGTCTACAGATGC
CCGGAAATCCTTCCCATGCTTTGACGAGCCAGCCATGAAGGCCACGTTC
AACATCACTCTCATCCACCCTAACAACCTCACGGCCCTGTCCAATATGC
CGCCCAAAGGTTCCAGCACCCCACTTGCAGAAGACCCCAACTGGTCTGT
CACTGAGTTCGAAACCACACCTGTGATGTCCACGTACCTTCTGGCCTAC
ATCGTGAGCGAGTTCCAGAGCGTGAATGAAACGGCCCAAAATGGCGTCC
TGATCCGGATCTGGGCTCGGCCTAATGCAATTGCAGAGGGCCATGGCAT
GTATGCCCTGAATGTGACAGGTCCCATCCTAAACTTCTTTGCCAATCAT
TATAATACACCCTACCCACTCCCCAAATCCGACCAGATTGCCTTGCCCG
ACTTCAATGCCGGTGCCATGGAGAACTGGGGGCTGGTGACCTACCGGGA
GAACGCGCTGCTGTTTGACCCACAGTCCTCCTCCATCAGCAACAAAGAG
CGAGTTGTCACTGTGATTGCTCACGAGCTGGCCCACCAGTGGTTTGGCA
ACCTGGTGACCCTGGCCTGGTGGAATGACCTGTGGCTGAATGAGGGCTT
TGCCTCCTATGTGGAGTACCTGGGTGCTGACCACGCAGAGCCCACCTGG
AATCTGAAAGACCTCATCGTGCCAGGCGACGTGTACCGAGTGATGGCTG
TGGATGCTCTGGCTTCCTCCCACCCGCTGACCACCCCTGCTGAGGAGGT
CAACACACCTGCCCAGATCAGCGAGATGTTTGACTCCATCTCCTACAGC
AAGGGAGCCTCGGTTATCAGGATGCTCTCCAACTTCCTGACTGAGGACC
TGTTCAAGGAGGGCCTGGCGTCCTACTTGCATGCCTTTGCCTATCAGAA
CACCACCTACCTGGACCTGTGGGAGCACCTGCAGAAGGCTGTGGATGCT
CAGACGTCCATCAGGCTGCCAGACACTGTGAGAGCCATCATGGATCGAT
GGACCCTGCAGATGGGCTTCCCCGTCATCACCGTGGACACCAAGACAGG
AAACATCTCACAGAAGCACTTCCTCCTCGACTCCGAATCCAACGTCACC
CGCTCCTCAGCGTTCGACTACCTCTGGATTGTTCCCATCTCATCTATTA
AAAATGGTGTGATGCAGGATCACTACTGGCTGCGGGATGTTTCCCAAGC
CCAGAATGATTTGTTCAAAACCGCATCGGACGATTGGGTCTTGCTGAAC
ATCAACGTGACAGGCTATTTCCAGGTGAACTACGACGAGGACAACTGGA
GGATGATTCAGCATCAGCTGCAGACAAACCTGTCGGTCATCCCTGTCAT
CAATCGGGCTCAGGTCATCTACGACAGCTTCAACCTGGCCACTGCCCAC
ATGGTCCCTGTCACCCTGGCTCTGGACAACACCCTCTTCCTGAACGGAG
AGAAAGAGTACATGCCCTGGCAGGCCGCCCTGAGCAGCCTGAGCTACTT
CAGCCTCATGTTCGACCGCTCCGAGGTCTATGGCCCCATGAAGAAATAC
CTCAGGAAGCAGGTCGAACCCCTCGCCCAACATTTCGAAACTCTCACTA
AAAACTGGACCGAGCGCCCAGAAAATCTGATGGACCAGTACAGTGAGAT
TAATGCCATCAGCACTGCCTGCTCCAATGGATTGCCTCAATGTGAGAAT
CTGGCCAAGACCCTTTTCGACCAGTGGATGAGCGACCCAGAAAATAACC
CGATCCACCCCAACCTGCGGTCCACCATCTACTGCAATGCCATAGCCCA
GGGCGGCCAGGACCAGTGGGACTTTGCCTGGGGGCAGTTACAACAAGCC
CAGCTGGTAAATGAGGCCGACAAACTCCGCTCAGCGCTGGCCTGCAGCA
ACGAGGTCTGGCTCCTGAACAGGTACCTGGGTTACACCCTGAACCCGGA
CCTCATTCGGAAGCAAGACGCCACCTCCACTATTAACAGCATTGCCAGC
AATGTCATCGGGCAGCCTCTGGCCTGGGATTTTGTCCAGAGCAACTGGA
AGAAGCTCTTTCAGGACTATGGCGGTGGTTCCTTCTCCTTCTCCAACCT
CATCCAGGGTGTGACCCGAAGATTCTCCTCTGAGTTTGAGCTGCAGCAG
CTGGAGCAGTTCAAGAAGAACAACATGGATGTGGGCTTCGGCTCCGGCA
CCCGGGCTCTGGAGCAAGCCCTGGAGAAGACCAAGGCCAACATCAAGTG
GGTGAAGGAGAACAAGGAGGTGGTGTTGAATTGGTTCATAGAGCACAGC
TAA;
14. Nucleotide sequence of E16 in FIG. 5
(SEQ ID NO: 14)
GTCGAACCCCTCTTCCAACATTTCGAAACTCTCACTA;
15. Nucleotide sequence of HDR template in FIG. 5
(SEQ ID NO: 15)
GTCGAACCCCTCGCCCAACATTTCGAAACTCTCACTA;
16. Nucleotide sequence of IPI-21-WT in FIG. 6
(SEQ ID NO: 16)
ACCCCTCTTCCAACATTTCGAAACTCTC;
17. Nucleotide sequence of IPI-21-727PE in FIG. 6
(SEQ ID NO: 17)
ACCCCTCGCCCAACATTTCGAAACTCTC;
18. Nucleotide sequence of PEF-WT in FIG. 12
(SEQ ID NO: 18)
ACCCCTCTTCCAACATTTCGAAACTCTC;
19. Nucleotide sequence of PEF-727PE in FIG. 12
(SEQ ID NO: 19)
ACCCCTCGCCCAACATTTCGAAACTCTC;
20. Nucleotide sequence of WT in FIG. 13
(SEQ ID NO: 20)
GTCGAACCCCTCTTCCAACATTTCGAAACTCTCACTA;
21. Nucleotide sequence of PIG-727PE in FIG. 13
(SEQ ID NO: 21)
GTCGAACCCCTCGCCCAACATTTCGAAACTCTCACTA.

1-10. (canceled)

11. A method for constructing a cell with a pAPN protein mutation comprising the steps of:
mutating phenylalanine to alanine at position 727 in a wild-type pAPN protein in a cell of interest to obtain the cell comprising the pAPN protein mutation;
wherein the step of mutating phenylalanine to alanine at position 727 in the wild-type pAPN protein comprises using a CRISPR system to introduce alanine at position 727 while maintaining all other amino acid residues found in the wild-type pAPN protein.

12-21. (canceled)

22. The method according to claim 11, wherein
the CRISPR system comprises sgRNA1, sgRNA2, and a donor DNA for homologous recombination;
wherein a nucleotide sequence of a target of the sgRNA1 is encoded by SEQ ID NO: 1;
wherein a nucleotide sequence of a target of the sgRNA2 is encoded by SEQ ID NO: 2;
wherein a nucleotide sequence of a donor DNA for homologous recombination is encoded by SEQ ID NO: 3; and
further wherein the wild-type pAPN protein is encoded by SEQ ID NO: 12.


SEQUENCE LISTING (21 sequences)

SEQ ID NO: 1 - DNA, 20 bp, synthetic construct
  source 1..20: mol_type = other DNA
ctagaaatacctcaggaagc

SEQ ID NO: 2 - DNA, 20 bp, synthetic construct
  source 1..20: mol_type = other DNA
gtttcgaaatgttggaagag

SEQ ID NO: 3 - DNA, 1000 bp, synthetic construct
  source 1..1000: mol_type = other DNA
cctttgagcacagtctggccttgtgcgaggcctttagcctctggcctcttgctcctgtagccattagctcttgctacatctgcccacccacatcagaggctccatgggtctccagatgactcaggcatgagtctcttctttgaagctatttttagggctgcatcctcggcatgtggaggttcccaagctaggggttgaatcggagctgtagccgccagcctacaccacagccacagcaacacgggatccgagccacatctgcgacctacaccacagctcacagcaatgccagatccttaacccactgagtggggccagggttgaacccatgtcctcatgtttcccagtcagattcgtttctgctgtgccatgacgggaactctggaacttcctctttgaagctctttatgttttgttcttgttttttgtttttgtttttctagaaatacctcaggaagcaagtcgaacccctcgcccaacatttcgaaactctcactaaaaactggaccgagcgcccagaaaatctgatggaccagtgagtatgagctcgcttggtctggagatcatgggtggtgcaggtagcctgacctgggggcccatagcaagtccagcagcatcctctctggagctcccaactcctggccggaccagggccacagtcagggagagcgacccctcccaaccccactcccggccccaggagtagggactctgctctgaggctctgtgtggcctatgaaccatctggcctctttgggcaaaggaccaaactgaacctctgagggtccctcacccgcatggtgaggttctaggtgttaaagctggggctggagcctgtgccagccctccccaggctgcccaagggcaagaagcaaagaagggaacccaaaggtggctggtgggctatacctgcagagtgcgggtctgcctccctgttgggagttgtgtgtcagcaggggagtcttggtcagcgtcaggtccaggcgtgctgacagagtgt

SEQ ID NO: 4 - DNA, 25 bp, synthetic construct
  source 1..25: mol_type = other DNA
caccgctagaaatacctcaggaagc

SEQ ID NO: 5 - DNA, 25 bp, synthetic construct
  source 1..25: mol_type = other DNA
aaacgcttcctgaggtatttctagc

SEQ ID NO: 6 - DNA, 24 bp, synthetic construct
  source 1..24: mol_type = other DNA
caccgtttcgaaatgttggaagag

SEQ ID NO: 7 - DNA, 24 bp, synthetic construct
  source 1..24: mol_type = other DNA
aaacctcttccaacatttcgaaac

SEQ ID NO: 8 - DNA, 20 bp, synthetic construct
  source 1..20: mol_type = other DNA
caaggatttgtggaggagaa

SEQ ID NO: 9 - DNA, 18 bp, synthetic construct
  source 1..18: mol_type = other DNA
gctgagcggagtttgtcg

SEQ ID NO: 10 - protein, 963 aa, synthetic construct
MAKGFYISKALGILGILLGVAAVATIIALSVVYAQEKNKNAEHVPQAPTSPTITTTAAITLDQSKPWNRYRLPTTLLPDSYNVTLRPYLTPNADGLYIFKGKSIVRFICQEPTDVIIIHSKKLNYTTQGHMVVLRGVGDSQVPEIDRTELVELTEYLVVHLKGSLQPGHMYEMESEFQGELADDLAGFYRSEYMEGNVKKVLATTQMQSTDARKSFPCFDEPAMKATFNITLIHPNNLTALSNMPPKGSSTPLAEDPNWSVTEFETTPVMSTYLLAYIVSEFQSVNETAQNGVLIRIWARPNAIAEGHGMYALNVTGPILNFFANHYNTPYPLPKSDQIALPDFNAGAMENWGLVTYRENALLFDPQSSSISNKERVVTVIAHELAHQWFGNLVTLAWWNDLWLNEGFASYVEYLGADHAEPTWNLKDLIVPGDVYRVMAVDALASSHPLTTPAEEVNTPAQISEMFDSISYSKGASVIRMLSNFLTEDLFKEGLASYLHAFAYQNTTYLDLWEHLQKAVDAQTSIRLPDTVRAIMDRWTLQMGFPVITVDTKTGNISQKHFLLDSESNVTRSSAFDYLWIVPISSIKNGVMQDHYWLRDVSQAQNDLFKTASDDWVLLNINVTGYFQVNYDEDNWRMIQHQLQTNLSVIPVINRAQVIYDSFNLATAHMVPVTLALDNTLFLNGEKEYMPWQAALSSLSYFSLMFDRSEVYGPMKKYLRKQVEPLFQHFETLTKNWTERPENLMDQYSEINAISTACSNGLPQCENLAKTLFDQWMSDPENNPIHPNLRSTIYCNAIAQGGQDQWDFAWGQLQQAQLVNEADKLRSALACSNEVWLLNRYLGYTLNPDLIRKQDATSTINSIASNVIGQPLAWDFVQSNWKKLFQDYGGGSFSFSNLIQGVTRRFSSEFELQQLEQFKKNNMDVGFGSGTRALEQALEKTKANIKWVKENKEVVLNWFIEHS

SEQ ID NO: 11 - DNA, 21 bp, synthetic construct
  source 1..21: mol_type = other DNA
gagggcctatttcccatgatt

SEQ ID NO: 12 - DNA, 2892 bp, synthetic construct
  source 1..2892: mol_type = other DNA
atggccaagggattctacatttccaaggccctgggcatcctgggcatcctcctcggcgtggcggccgtggccaccatcatcgctctgtctgtggtgtacgcccaggagaagaacaagaatgccgagcatgtcccccaggcccccacgtcgcccaccatcaccaccacagccgccatcaccttggaccagagcaagccgtggaaccggtaccgcctacccacaacgctgttgcctgattcctacaacgtgacgctgagaccctacctcactcccaacgcggatggcctgtacatcttcaagggcaaaagcatcgtccgcttcatctgccaggagcccaccgatgtcatcatcatccatagcaagaagctcaactacaccacccaggggcacatggtggtcctgcggggcgtgggggactcccaggtcccagagatcgacaggactgagctggtagagctcactgagtacctggtggtccacctcaagggctcgctgcagcccggccacatgtacgagatggagagtgaattccagggggaacttgccgacgacctggcaggcttctaccgcagcgagtacatggagggcaacgtcaaaaaggtgctggccacgacacagatgcagtctacagatgcccggaaatccttcccatgctttgacgagccagccatgaaggccacgttcaacatcactctcatccaccctaacaacctcacggccctgtccaatatgccgcccaaaggttccagcaccccacttgcagaagaccccaactggtctgtcactgagttcgaaaccacacctgtgatgtccacgtaccttctggcctacatcgtgagcgagttccagagcgtgaatgaaacggcccaaaatggcgtcctgatccggatctgggctcggcctaatgcaattgcagagggccatggcatgtatgccctgaatgtgacaggtcccatcctaaacttctttgccaatcattataatacaccctacccactccccaaatccgaccagattgccttgcccgacttcaatgccggtgccatggagaactgggggctggtgacctaccgggagaacgcgctgctgtttgacccacagtcctcctccatcagcaacaaagagcgagttgtcactgtgattgctcacgagctggcccaccagtggtttggcaacctggtgaccctggcctggtggaatgacctgtggctgaatgagggctttgcctcctatgtggagtacctgggtgctgaccacgcagagcccacctggaatctgaaagacctcatcgtgccaggcgacgtgtaccgagtgatggctgtggatgctctggcttcctcccacccgctgaccacccctgctgaggaggtcaacacacctgcccagatcagcgagatgtttgactccatctcctacagcaagggagcctcggttatcaggatgctctccaacttcctgactgaggacctgttcaaggagggcctggcgtcctacttgcatgcctttgcctatcagaacaccacctacctggacctgtgggagcacctgcagaaggctgtggatgctcagacgtccatcaggctgccagacactgtgagagccatcatggatcgatggaccctgcagatgggcttccccgtcatcaccgtggacaccaagacaggaaacatctcacagaagcacttcctcctcgactccgaatccaacgtcacccgctcctcagcgttcgactacctctggattgttcccatctcatctattaaaaatggtgtgatgcaggatcactactggctgcgggatgtttcccaagcccagaatgatttgttcaaaaccgcatcggacgattgggtcttgctgaacatcaacgtgacaggctatttccaggtgaactacgacgaggacaactggaggatgattcagcatcagctgcagacaaacctgtcggtcatccctgtcatcaatcgggctcaggtcatctacgacagcttcaacctggccactgcccacatggtccctgtcaccctggctctggacaacaccctcttcctgaacggagagaaagagtacatgccctggcaggccgccctgagcagcctgagctacttcagcctcatgttcgaccgctccgaggtctatggccccatgaagaaatacctcaggaagcaggtcgaacccctcttccaacatttcgaaactctcactaaaaactggaccgagcgcccagaaaatctgatggaccagtacagtgagattaatgccatcagcactgcctgctccaatggattgcctcaatgtgagaatctggccaagacccttttcgaccagtggatgagcgacccagaaaataacccgatccaccccaacctgcggtccaccatctactgcaatgccatagcccagggcggccaggaccagtgggactttgcctgggggcagttacaacaagcccagctggtaaatgaggccgacaaactccgctcagcgctggcctgcagcaacgaggtctggctcctgaacaggtacctgggttacaccctgaacccggacctcattcggaagcaagacgccacctccactattaacagcattgccagcaatgtcatcgggcagcctctggcctgggattttgtccagagcaactggaagaagctctttcaggactatggcggtggttccttctccttctccaacctcatccagggtgtgacccgaagattctcctctgagtttgagctgcagcagctggagcagttcaagaagaacaacatggatgtgggcttcggctccggcacccgggctctggagcaagccctggagaagaccaaggccaacatcaagtgggtgaaggagaacaaggaggtggtgttgaattggttcatagagcacagctaa

SEQ ID NO: 13 - DNA, 2892 bp, synthetic construct
  source 1..2892: mol_type = other DNA
atggccaagggattctacatttccaaggccctgggcatcctgggcatcctcctcggcgtggcggccgtggccaccatcatcgctctgtctgtggtgtacgcccaggagaagaacaagaatgccgagcatgtcccccaggcccccacgtcgcccaccatcaccaccacagccgccatcaccttggaccagagcaagccgtggaaccggtaccgcctacccacaacgctgttgcctgattcctacaacgtgacgctgagaccctacctcactcccaacgcggatggcctgtacatcttcaagggcaaaagcatcgtccgcttcatctgccaggagcccaccgatgtcatcatcatccatagcaagaagctcaactacaccacccaggggcacatggtggtcctgcggggcgtgggggactcccaggtcccagagatcgacaggactgagctggtagagctcactgagtacctggtggtccacctcaagggctcgctgcagcccggccacatgtacgagatggagagtgaattccagggggaacttgccgacgacctggcaggcttctaccgcagcgagtacatggagggcaacgtcaaaaaggtgctggccacgacacagatgcagtctacagatgcccggaaatccttcccatgctttgacgagccagccatgaaggccacgttcaacatcactctcatccaccctaacaacctcacggccctgtccaatatgccgcccaaaggttccagcaccccacttgcagaagaccccaactggtctgtcactgagttcgaaaccacacctgtgatgtccacgtaccttctggcctacatcgtgagcgagttccagagcgtgaatgaaacggcccaaaatggcgtcctgatccggatctgggctcggcctaatgcaattgcagagggccatggcatgtatgccctgaatgtgacaggtcccatcctaaacttctttgccaatcattataatacaccctacccactccccaaatccgaccagattgccttgcccgacttcaatgccggtgccatggagaactgggggctggtgacctaccgggagaacgcgctgctgtttgacccacagtcctcctccatcagcaacaaagagcgagttgtcactgtgattgctcacgagctggcccaccagtggtttggcaacctggtgaccctggcctggtggaatgacctgtggctgaatgagggctttgcctcctatgtggagtacctgggtgctgaccacgcagagcccacctggaatctgaaagacctcatcgtgccaggcgacgtgtaccgagtgatggctgtggatgctctggcttcctcccacccgctgaccacccctgctgaggaggtcaacacacctgcccagatcagcgagatgtttgactccatctcctacagcaagggagcctcggttatcaggatgctctccaacttcctgactgaggacctgttcaaggagggcctggcgtcctacttgcatgcctttgcctatcagaacaccacctacctggacctgtgggagcacctgcagaaggctgtggatgctcagacgtccatcaggctgccagacactgtgagagccatcatggatcgatggaccctgcagatgggcttccccgtcatcaccgtggacaccaagacaggaaacatctcacagaagcacttcctcctcgactccgaatccaacgtcacccgctcctcagcgttcgactacctctggattgttcccatctcatctattaaaaatggtgtgatgcaggatcactactggctgcgggatgtttcccaagcccagaatgatttgttcaaaaccgcatcggacgattgggtcttgctgaacatcaacgtgacaggctatttccaggtgaactacgacgaggacaactggaggatgattcagcatcagctgcagacaaacctgtcggtcatccctgtcatcaatcgggctcaggtcatctacgacagcttcaacctggccactgcccacatggtccctgtcaccctggctctggacaacaccctcttcctgaacggagagaaagagtacatgccctggcaggccgccctgagcagcctgagctacttcagcctcatgttcgaccgctccgaggtctatggccccatgaagaaatacctcaggaagcaggtcgaacccctcgcccaacatttcgaaactctcactaaaaactggaccgagcgcccagaaaatctgatggaccagtacagtgagattaatgccatcagcactgcctgctccaatggattgcctcaatgtgagaatctggccaagacccttttcgaccagtggatgagcgacccagaaaataacccgatccaccccaacctgcggtccaccatctactgcaatgccatagcccagggcggccaggaccagtgggactttgcctgggggcagttacaacaagcccagctggtaaatgaggccgacaaactccgctcagcgctggcctgcagcaacgaggtctggctcctgaacaggtacctgggttacaccctgaacccggacctcattcggaagcaagacgccacctccactattaacagcattgccagcaatgtcatcgggcagcctctggcctgggattttgtccagagcaactggaagaagctctttcaggactatggcggtggttccttctccttctccaacctcatccagggtgtgacccgaagattctcctctgagtttgagctgcagcagctggagcagttcaagaagaacaacatggatgtgggcttcggctccggcacccgggctctggagcaagccctggagaagaccaaggccaacatcaagtgggtgaaggagaacaaggaggtggtgttgaattggttcatagagcacagctaa

SEQ ID NO: 14 - DNA, 37 bp, synthetic construct
  source 1..37: mol_type = other DNA
gtcgaacccctcttccaacatttcgaaactctcacta

SEQ ID NO: 15 - DNA, 37 bp, synthetic construct
  source 1..37: mol_type = other DNA
gtcgaacccctcgcccaacatttcgaaactctcacta

SEQ ID NO: 16 - DNA, 28 bp, synthetic construct
  source 1..28: mol_type = other DNA
acccctcttccaacatttcgaaactctc

SEQ ID NO: 17 - DNA, 28 bp, synthetic construct
  source 1..28: mol_type = other DNA
acccctcgcccaacatttcgaaactctc

SEQ ID NO: 18 - DNA, 28 bp, synthetic construct
  source 1..28: mol_type = other DNA
acccctcttccaacatttcgaaactctc

SEQ ID NO: 19 - DNA, 28 bp, synthetic construct
  source 1..28: mol_type = other DNA
acccctcgcccaacatttcgaaactctc

SEQ ID NO: 20 - DNA, 37 bp, synthetic construct; identical to SEQ ID NO: 14
  source 1..37: mol_type = other DNA

SEQ ID NO: 21 - DNA, 37 bp, synthetic construct; identical to SEQ ID NO: 15
  source 1..37: mol_type = other DNA